(19) United States
(12) Patent Application Publication
(10) Pub. No.: US 2012/0095450 A1
(43) Pub. Date: Apr. 19, 2012

(54) Elongated and retractable sterile marker apparatus

(76) Inventors: Timothy W. Morgan, Paragould, AR (US); Thomas R. Smith, Blytheville, AR (US)

(21) Appl. No.: 12/925,191

(22) Filed: Oct. 15, 2010

(51) Int. Cl. A61B 19/00

(52) U.S. Cl. 606/1

(57) ABSTRACT

An elongated and retractable sterile marker apparatus includes a handle assembly, an elongated marker operating assembly a top portion of which is connected to the handle assembly, and a sterile marker assembly which is connected to a bottom portion of the elongated marker operating assembly. Preferably, the sterile marker assembly includes a felt marker tip. The elongated and retractable sterile marker apparatus is particularly useful in pain management procedures to mark needle locations. Preferably, the handle assembly includes a pair of finger-reception members which are connected to an outside portion of the elongated marker operating assembly. A thumb-reception member is received in the elongated marker operating assembly for operating the elongated marker operating assembly. Preferably, a return spring is provided to return the tip of the sterile marker portion into a marker unit housing when the tip is no longer being used for marking.

### BACKGROUND OF THE INVENTION

1. Field of the Invention

The present invention relates generally to marking devices, and, more particularly, to marking devices especially adapted for marking human skin.

2. Description of the Prior Art

Marking devices for marking the skin of living persons or animals is well known in the art. Throughout the years, a number of innovations have been developed relating to marking skin, and the following U.S. Pat. Nos. are representative of some of those innovations: 4,572,831, 5,960,802, 6,056,737, 6,729,786, 6,972,022.

The marking devices in all of the above-cited patents have one thing in common. They all require that a cover must be put the respective device to protect the marking portion; and the cover must be removed from the respective marking device to allow the marking portion to be used. And, to remove and replace the cover requires two-handed operation. Rather than require two-handed operation, it would be desirable if a skin marking device were provided that permits only one-handed operation.

In addition, all of the above-cited patents are subject to the possibility that the respective cover may be lost or misplaced or fall to the floor and become dirty. With such risks for a cover, the cover can be contaminated, and a contaminated cover can defeat the use of a sterile marker. In this respect, it would be desirable if a skin marking device were provided which does not include a removable cover that may be lost or misplaced or fall to the floor and become dirty.

Not only would it be desirable for a skin marking device to permit one-handed operation and to permit a marking portion to be exposed for marking the skin without removing a cover, it would also be desirable for the marking portion to be automatically protected when the user is no longer using the marking device.

There are times when a medical practitioner would like to mark a patient's skin when the patient's skin is in a field of radiation. However, the medical practitioner, in marking the patient's skin, would not want one's hand to be exposed to the same radiation field that the patient is exposed to. In this respect, it would be desirable for a marking device to have an elongated handle so that a medical practitioner can hold and use the marking device with the practitioner's hand outside of a radiation field while, at the same time, the patient's skin is exposed to the radiation field.

With use by a medical practitioner, it would be desirable for a skin marking device to include a sterile marking portion. Moreover, to preserve sterile or near sterile conditions in the marking portion, it would be desirable if the skin marking device permitted retraction of the marking portion into a sterile or near sterile housing when the marking portion was not in use.

Thus, while the foregoing body of prior art indicates it to be well known to use skin marking devices, the prior art described above does not teach or suggest an elongated and retractable sterile marker apparatus which has the following combination of desirable features: (1) permits only one-handed operation; (2) does not include a removable cover that may be lost or misplaced or fall to the floor and become dirty; (3) permits the marking portion to be automatically protected when the user is no longer using the marking device; (4) provides a marking device which has an elongated handle so that a medical practitioner can hold and use the marking device with the practitioner's hand outside of a radiation field while, at the same time, the patient's skin is exposed to the radiation field; (5) permits retraction of the marking portion into a sterile or near sterile housing when the marking portion is not in use. The foregoing desired characteristics are provided by the unique elongated and retractable sterile marker apparatus of the present invention as will be made apparent from the following description thereof. Other advantages of the present invention over the prior art also will be rendered evident.

### SUMMARY OF THE INVENTION

To achieve the foregoing and other advantages, the present invention, briefly described, provides an elongated and retractable sterile marker apparatus includes a handle assembly, an elongated marker operating assembly a top portion of which is connected to the handle assembly, and a sterile marker assembly which is connected to a bottom portion of the elongated marker operating assembly. Preferably, the sterile marker assembly includes a felt marker tip. The elongated and retractable sterile marker apparatus is particularly useful in pain management procedures to mark needle locations.

Preferably, the handle assembly includes a pair of finger-reception members which are connected to an outside portion of the elongated marker operating assembly. A thumb-reception member is received in the elongated marker operating assembly for operating the elongated marker operating assembly.

In addition, the handle assembly includes a reception sleeve, a top portion of which receives the thumb-reception member. A ram unit is received in a bottom portion of the reception sleeve. The ram unit drives the elongated marker operating assembly for operating the sterile marker assembly.

More specifically, the ram unit includes a top portion which includes a thumb-reception-member receiving well which receives a bottom portion of the thumb-reception member, and the ram unit includes a bottom ram head for driving the elongated marker operating assembly.

Preferably, the elongated marker operating assembly includes an elongated housing, and an elongated marker driver is in contact with the handle assembly.

Preferably, the sterile marker assembly includes a marker unit housing which is connected to a bottom end of the elongated marker operating assembly. A marker unit is retained in the marker unit housing, and a return spring is interposed between the elongated marker operating assembly and the marker unit for pulling the marker unit toward the elongated marker operating assembly when the handle assembly is no longer actuated.

Preferably, the marker unit includes a marker retention portion which is received in a bottom portion of the elongated marker operating assembly, and a sterile marker portion is connected to the marker retention portion.

The above brief description sets forth rather broadly the more important features of the present invention in order that the detailed description thereof that follows may be better understood, and in order that the present contributions to the art may be better appreciated. There are, of course, additional features of the invention that will be described hereinafter and which will be for the subject matter of the claims appended hereto.

In this respect, before explaining a preferred embodiment of the invention in detail, it is understood that the invention is not limited in its application to the details of the construction and to the arrangements of the components set forth in the following description or illustrated in the drawings. The invention is capable of other embodiments and of being practiced and carried out in various ways. Also, it is to be understood, that the phraseology and terminology employed herein are for the purpose of description and should not be regarded as limiting.

As such, those skilled in the art will appreciate that the conception, upon which disclosure is based, may readily be utilized as a basis for designing other structures, methods, and systems for carrying out the several purposes of the present invention. It is important, therefore, that the claims be regarded as including such equivalent constructions insofar as they do not depart from the spirit and scope of the present invention.

It is therefore an object of the present invention to provide a new and improved elongated and retractable sterile marker apparatus which has all of the advantages of the prior art and none of the disadvantages.

It is another object of the present invention to provide a new and improved elongated and retractable sterile marker apparatus which may be easily and efficiently manufactured and marketed.

It is a further object of the present invention to provide a new and improved elongated and retractable sterile marker apparatus which is of durable and reliable construction.

An even further object of the present invention is to provide a new and improved elongated and retractable sterile marker apparatus which is susceptible of a low cost of manufacture with regard to both materials and labor, and which accordingly is then susceptible of low prices of sale to the consuming public, thereby making such elongated and retractable sterile marker apparatus available to the buying public.

Still yet a further object of the present invention is to provide a new and improved elongated and retractable sterile marker apparatus which permits only one-handed operation.

Still another object of the present invention is to provide a new and improved elongated and retractable sterile marker apparatus that does not include a removable cover that may be lost or misplaced or fall to the floor and become dirty.

Yet another object of the present invention is to provide a new and improved elongated and retractable sterile marker apparatus which permits the marking portion to be automatically protected when the user is no longer using the marking device.

Even another object of the present invention is to provide a new and improved elongated and retractable sterile marker apparatus that provides a marking device which has an elongated handle so that a medical practitioner can hold and use the marking device with the practitioner's hand outside of a radiation field while, at the same time, the patient's skin is exposed to the radiation field.

Still a further object of the present invention is to provide a new and improved elongated and retractable sterile marker apparatus which permits retraction of the marking portion into a sterile or near sterile housing when the marking portion is not in use.

These together with still other objects of the invention, along with the various features of novelty which characterize the invention, are pointed out with particularity in the claims annexed to and forming a part of this disclosure. For a better understanding of the invention, its operating advantages and the specific objects attained by its uses, reference should be had to the accompanying drawings and descriptive matter in which there are illustrated preferred embodiments of the invention.

### BRIEF DESCRIPTION OF THE DRAWINGS

The invention will be better understood and the above objects as well as objects other than those set forth above will become more apparent after a study of the following detailed description thereof. Such description makes reference to the annexed drawing wherein:

FIG. 1 is a front view showing a preferred embodiment of the elongated and retractable sterile marker apparatus of the invention.

FIG. 2 is an enlarged side view of a top portion of the embodiment of the elongated and retractable sterile marker apparatus shown in FIG. 1 taken along line 2-2 of FIG. 1.

FIG. 3 is an enlarged view of a bottom portion of the embodiment of the elongated and retractable sterile marker apparatus of FIG. 1 that is contained in circled region 3 thereof.

FIG. 4 is a top view of the embodiment of the invention shown in FIG. 2 taken along line 4-4 thereof.

FIG. 5 is an exploded view of a portion of the embodiment of the invention shown in FIG. 3.

FIG. 6 is an exploded view of a portion of the embodiment of the invention shown in FIG. 2.

### DESCRIPTION OF THE PREFERRED EMBODIMENT

With reference to the drawings, a new and improved elongated and retractable sterile marker apparatus embodying the principles and concepts of the present invention will be described.

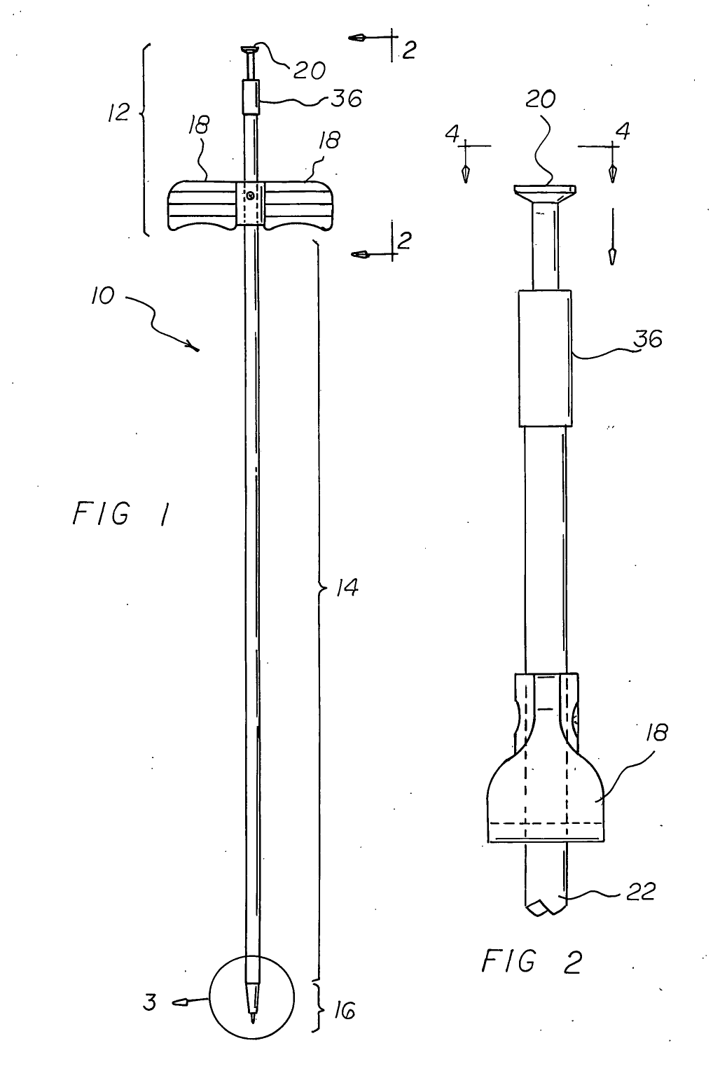

Turning to FIGS. 1-6, there is shown a preferred embodiment of the elongated and retractable sterile marker apparatus of the invention generally designated by reference numeral 10. In each of the figures, reference numerals are shown that correspond to like reference numerals that designate like elements shown in other figures.

In the preferred embodiment, elongated and retractable sterile marker apparatus 10 includes a handle assembly 12, an elongated marker operating assembly 14 a top portion of which is connected to the handle assembly 12, and a sterile marker assembly 16 which is connected to a bottom portion of the elongated marker operating assembly 14.

Preferably, the handle assembly 12 includes a pair of finger-reception members 18 which are connected to an outside portion of the elongated marker operating assembly 14. A thumb-reception member 20 is received in the elongated marker operating assembly 14 for operating the elongated marker operating assembly 14.

In addition, the handle assembly 12 includes a reception sleeve 36, a top portion of which receives the thumb-reception member 20. A ram unit 38 is received in a bottom portion of the reception sleeve 36. The ram unit 38 drives the elongated marker operating assembly 14 for operating the sterile marker assembly 16.

More specifically, the ram unit 38 includes a top portion which includes a thumb-reception-member receiving well 40 which receives a bottom portion of the thumb-reception member 20, and the ram unit 38 includes a bottom ram head 42 for driving the elongated marker operating assembly 14.

Preferably, the elongated marker operating assembly 14 includes an elongated housing 22, and an elongated marker driver 24 is in contact with the handle assembly 12. That is, the elongated marker driver 24 is driven by the thumb-reception member 20 in the handle assembly 12.

Preferably, the sterile marker assembly 16 includes a marker unit housing 26 which is connected to a bottom end of the elongated marker operating assembly 14. A marker unit is retained in the marker unit housing 26, and a return spring 28 is interposed between the elongated marker operating assembly 14 and the marker unit for pulling the marker unit toward the elongated marker operating assembly 14 when the handle assembly 12 is no longer actuated.

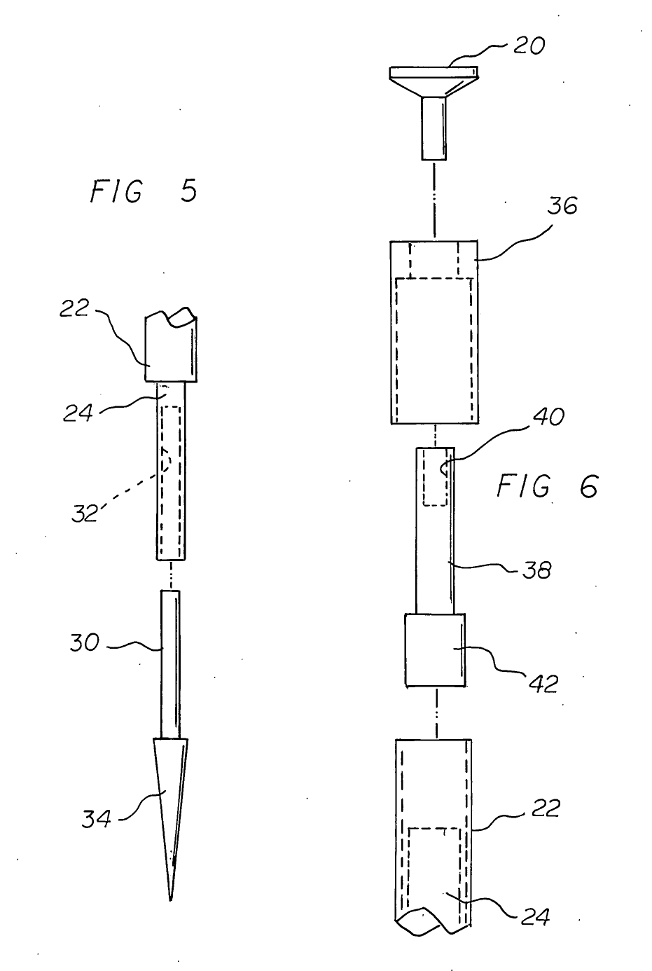

Preferably, the marker unit includes a marker retention portion 30 which is received in a bottom portion of the elongated marker operating assembly 14, and a sterile marker portion 34 is connected to the marker retention portion 30. More specifically, the marker retention portion 30 is received in a retention well 32 in the bottom end of the elongated marker driver 24, as shown in FIG. 5. Preferably, the sterile marker portion 34 employs a felt tip.

The elongated and retractable sterile marker apparatus 10 is particularly useful in pain management procedures to mark needle locations.

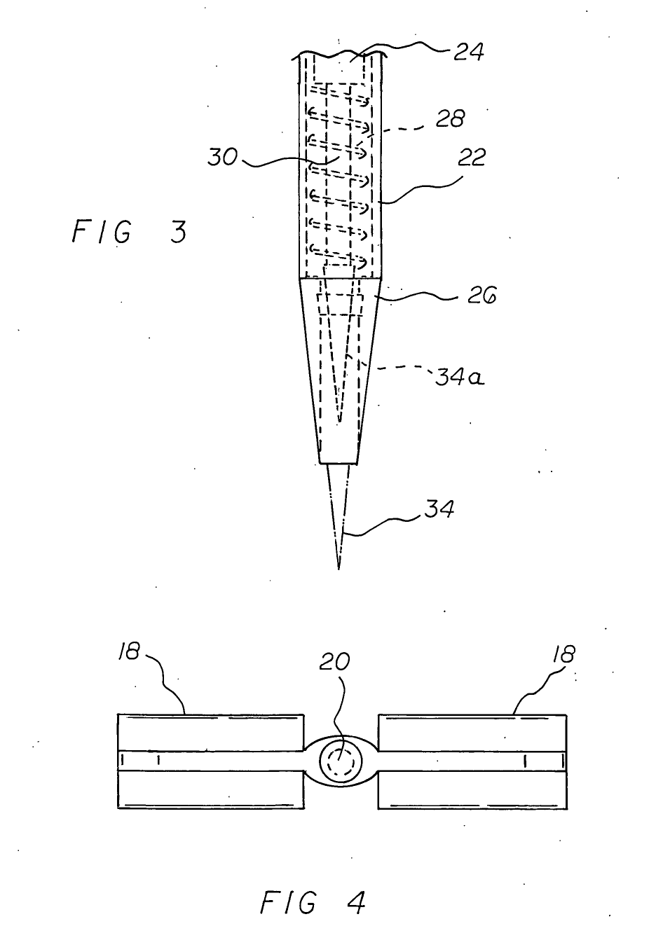

In operation, the sterile marker portion 34 is normally retained inside the marker unit housing 26, as shown by element 34a in FIG. 3. When it is desired to extend the sterile marker portion 34 to the use position, as shown by element 34 in FIG. 3, with one hand. A user grasps the handle assembly 12. More specifically, the user places some of one's fingers of the bottom side of the finger-reception members 18. Also, the user places one's thumb on the thumb-reception member 20.

Then, the user presses down on the thumb-reception member 20. As this is done, the pushing force of the user's thumb is transferred to the ram head 42 of the ram unit 38. Force from the ram head 42 is transferred to the elongated marker driver 24 which transfers the pushing force to the top of the sterile marker portion 34, causing the tip of the sterile marker portion 34 to extend outside the marker unit housing 26, as shown in FIG. 3.

It is noted that when the pushing force of the user's thumb is transferred from the elongated marker driver 24 to the marker retention portion 30 to extend the tip of the sterile marker portion 34 outside of the marker unit housing 26, the return spring 28 is compressed between the bottom of the elongated marker driver 24 and the top of the sterile marker portion 34. Subsequently, when the user releases one's thumb from the thumb-reception member 20, the compression of the return spring 28 is released, and the return spring 28 decompresses to its normal state, whereby the tip end of the sterile marker portion 34 is retracted back into the marker unit housing 26. To facilitate the retention of sterile characteristics, the sterile marker portion 34 does not lock in the extended position.

As stated above, the elongated and retractable sterile marker apparatus 10 is particularly useful in pain management procedures to mark needle locations. The elongated and retractable sterile marker apparatus 10 can be approximately 17 inches long so that a physician's hands would be outside of a radiation field.

There are two different versions of the elongated and retractable sterile marker apparatus 10. With a first version, the elongated and retractable sterile marker apparatus 10 is made from durable materials, such as metal. In this respect, the first version is reusable. However, the reusable has a disposable sterile marker portion 34.

With a second version of the elongated and retractable sterile marker apparatus 10, the entire apparatus is disposable. In this respect, the entire elongated and retractable sterile marker apparatus 10 is made from disposable materials. However, the disposable version has a small metal tip so as to visible in an X-ray.

The components of the elongated and retractable sterile marker apparatus of the invention can be made from inexpensive and durable metal and plastic materials.

The foregoing detailed description is considered as illustrative only of the principles of the invention. Numerous modifications and changes will readily occur to those skilled in the art and therefore, it is not desired to limit the invention to the exact construction and operation shown and described. Accordingly, all suitable modifications and equivalents falling within the broad scope of the subject matter described above may be resorted to in carrying out the present invention.

As to the manner of usage and operation of the instant invention, the same is apparent from the above disclosure, and accordingly, no further discussion relative to the manner of usage and operation need be provided.

It is apparent from the above that the present invention accomplishes all of the objects set forth by providing a new and improved elongated and retractable sterile marker apparatus that is low in cost, relatively simple in design and operation, and which may advantageously be used which permits only one-handed operation. With the invention, an elongated and retractable sterile marker apparatus is provided which does not include a removable cover that may be lost or misplaced or fall to the floor and become dirty. With the invention, an elongated and retractable sterile marker apparatus is provided which permits the marking portion to be automatically protected when the user is no longer using the marking device. With the invention, an elongated and retractable sterile marker apparatus provides a marking device which has an elongated handle so that a medical practitioner can hold and use the marking device with the practitioner's hand outside of a radiation field while, at the same time, the patient's skin is exposed to the radiation field. With the invention, an elongated and retractable sterile marker apparatus is provided which permits retraction of the marking portion into a sterile or near sterile housing when the marking portion is not in use.

Thus, while the present invention has been shown in the drawings and fully described above with particularity and detail in connection with what is presently deemed to be the most practical and preferred embodiment(s) of the invention, it will be apparent to those of ordinary skill in the art that many modifications thereof may be made without departing from the principles and concepts set forth herein, including, but not limited to, variations in size, materials, shape, form, function and manner of operation, assembly and use.

Hence, the proper scope of the present invention should be determined only by the broadest interpretation of the appended claims so as to encompass all such modifications as well as all relationships equivalent to those illustrated in the drawings and described in the specification.

Finally, it will be appreciated that the purpose of the annexed Abstract is to enable the U.S. Patent and Trademark Office and the public generally, and especially the scientists, engineers and practitioners in the art who are not familiar with patent or legal terms or phraseology, to determine quickly from a cursory inspection the nature and essence of the technical disclosure of the application. Accordingly, the Abstract is neither intended to define the invention or the application, which only is measured by the claims, nor is it intended to be limiting as to the scope of the invention in any way.

What is claimed as being new and desired to be protected by LETTERS PATENT of the United States is as follows:

1. An elongated and retractable sterile marker apparatus, comprising:
a handle assembly,
an elongated marker operating assembly a top portion of which is connected to said handle assembly, and
a sterile marker assembly connected to a bottom portion of said elongated marker operating assembly.

2. The apparatus of claim 1 wherein said handle assembly includes:
a pair of finger-reception members connected to an outside portion of said elongated marker operating assembly, and
a thumb-reception member received in said elongated marker operating assembly for operating said elongated marker operating assembly.

3. The apparatus of claim 1 wherein said handle assembly includes:
a reception sleeve a top portion of which receives said thumb-reception member,
a ram unit received in a bottom portion of said reception sleeve, wherein said ram unit drives said elongated marker operating assembly for operating said sterile marker assembly.

4. The apparatus of claim 3 wherein:
said ram unit includes a top portion which includes a thumb-reception-member receiving well which receives a bottom portion of said thumb-reception member, and wherein said ram unit includes a bottom ram head for driving said elongated marker operating assembly.

5. The apparatus of claim 1 wherein said elongated marker operating assembly includes:
an elongated housing, and
an elongated marker driver in contact with said handle assembly.

6. The apparatus of claim 1 wherein said sterile marker assembly includes:
a marker unit housing connected to a bottom end of said elongated marker operating assembly,
a marker unit retained in said marker unit housing, and a return spring interposed between said elongated marker operating assembly and said marker unit for pulling said marker unit into said elongated marker operating assembly when said handle assembly is actuated.

7. The apparatus of claim 6 wherein said marker unit includes:
a marker retention portion which is received in a bottom portion of said elongated marker operating assembly, and
a sterile marker portion connected to said marker retention portion.
